(19) United States
(12) Patent Application Publication
(10) Pub. No.: US 2026/0007410 A1
(43) Pub. Date: Jan. 8, 2026

(54) GUIDE CATHETER WITH FLAT SEAL

(71) Applicant: BOSTON SCIENTIFIC SCIMED, INC., MAPLE GROVE, MN (US)

(72) Inventors: Aniket Kajal, kurukshetra (IN); Ryan Robert Davis, Plymouth, MN (US); Neeraj Kumar Sharma, New Delhi (IN); Peeyush Tomar, Muzaffarnagar (IN); Shubham Gupta, Gurgaon (IN)

(73) Assignee: BOSTON SCIENTIFIC SCIMED, INC., MAPLE GROVE, MN (US)

(21) Appl. No.: 19/257,967

(22) Filed: Jul. 2, 2025

Related U.S. Application Data
(60) Provisional application No. 63/666,959, filed on Jul. 2, 2024.

(51) Int. Cl. A61B 17/12; A61B 17/00; A61M 25/00; A61M 25/06

(52) U.S. Cl. CPC A61B 17/12122; A61B 17/00234; A61M 25/0097; A61M 25/0662; A61B 2017/00305; A61B 2017/00367; A61B 2017/1205; A61M 2210/125

(57) ABSTRACT

A guide catheter for accommodating a left atrial appendage closure (LAAC) device being advanced towards a left atrial appendage (LAA) includes an elongate shaft extending from a proximal region to a distal region and a guide catheter hub that is coupled to the proximal region of the elongate shaft. The guide catheter hub includes a hub body, a proximal cap that is secured to the hub body and that includes a proximal cap extension defining a lumen extending through the proximal cap extension. An actuation member is slidingly disposed within the hub body and includes a central bore defining a lumen extending through the actuation member and a handle extending radially outwardly from the central bore. A flat seal is disposed within the actuation member. Advancing the actuation member towards the proximal cap causes the proximal cap extension to extend through the flat seal.

### CROSS REFERENCE TO RELATED APPLICATIONS

This application claims the benefit of priority of U.S. Provisional Application No. 63/666,959 filed Jul. 2, 2024, the entire disclosure of which is hereby incorporated by reference.

### TECHNICAL FIELD

The disclosure relates generally to medical devices and more particularly to guide catheters including guide catheter hubs that provide a fluid-tight access through the guide catheter hub for other medical devices.

### BACKGROUND

A wide variety of intracorporeal medical devices have been developed for medical use, for example, intravascular use. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a guide catheter for accommodating a left atrial appendage closure (LAAC) device being advanced towards a left atrial appendage (LAA). The guide catheter includes an elongate shaft extending from a proximal region to a distal region and a guide catheter hub that is coupled to the proximal region of the elongate shaft. The guide catheter hub includes a hub body and a proximal cap that is secured to the hub body. The proximal cap includes a proximal cap extension that defines a lumen extending through the proximal cap extension. An actuation member is slidingly disposed within the hub body. The actuation member includes a central bore defining a lumen extending through the actuation member and a handle extending radially outwardly from the central bore. A flat seal is disposed within the actuation member. Advancing the actuation member towards the proximal cap causes the proximal cap extension to extend through the flat seal.

Alternatively or additionally, the actuation member may be movable between a first position in which the flat seal is closed and a second position in which the flat seal is open.

Alternatively or additionally, the actuation member may be biased to the first position.

Alternatively or additionally, the guide catheter may further include a spring that biases the actuation member to the first position.

Alternatively or additionally, resiliency of the flat seal may provide a biasing force to bias the actuation member to the first position.

Alternatively or additionally, the lumen extending through the proximal cap extension may be fluidly coupled to and axially aligned with the lumen extending through the actuation member when the actuation member is in the second position.

Alternatively or additionally, the guide catheter may further include a distal lumen extending through the hub body, and the distal lumen may be fluidly coupled to and axially aligned with the lumen extending through the actuation member.

Alternatively or additionally, the proximal cap may be adapted to provide a rotational lock with a dilator.

Another example may be found in a guide catheter for accommodating a left atrial appendage closure (LAAC) device being advanced towards a left atrial appendage (LAA). The guide catheter includes an elongate shaft extending from a proximal region to a distal region and a guide catheter hub that is coupled to the proximal region of the elongate shaft. The guide catheter hub includes a hub body and a proximal cap that is secured to the hub body. The proximal cap includes a proximal cap extension defining a lumen extending through the proximal cap extension. An actuation member slidingly disposed within the hub body. The actuation member includes a central bore defining a lumen extending through the actuation member that is axially aligned with the lumen extending through the proximal cap extension and a handle that extends radially outwardly from the central bore. A flat seal is disposed within the actuation member. The actuation member is biased to a closed position in which the flat seal prevents the lumen extending through the actuation member from being fluidly coupled with the lumen extending through the proximal cap extension.

Alternatively or additionally, advancing the actuation member towards the proximal cap may cause the proximal cap extension to extend through the flat seal and fluidly couple the lumen extending through the actuation member with the lumen extending through the proximal cap extension.

Alternatively or additionally, the guide catheter may further include a distal lumen extending through the hub body, and the distal lumen may be fluidly coupled to and axially aligned with the lumen extending through the actuation member.

Alternatively or additionally, the actuation member may be movable from a closed position in which the flat seal is closed to an open position in which the flat seal is open.

Alternatively or additionally, the actuation member may be adapted to return to the closed position when the handle is released.

Alternatively or additionally, resiliency of the flat seal may provide a biasing force that biases the actuation member to the closed position.

Alternatively or additionally, the guide catheter may further include a spring that biases the actuation member to the closed position.

Alternatively or additionally, the proximal cap may be adapted to provide a rotational lock with a second medical device extending through the guide catheter.

Another example may be found in a medical device. The medical device includes an elongate shaft extending from a proximal region to a distal region and a medical device hub that is coupled to the proximal region of the elongate shaft. The medical device hub includes a hub body including a distal lumen extending through the hub body and a proximal cap that is secured to the hub body. The proximal cap includes a proximal cap extension defining a lumen that extends through the proximal cap extension. An actuation member is slidingly disposed within the hub body and includes a central bore defining a lumen extending through the actuation member and a handle extending radially outwardly from the central bore. A flat seal is disposed within the actuation member. The actuation member is biased to a position in which the flat seal is closed. Advancing the actuation member towards the proximal cap causes the proximal cap extension to open the flat seal.

Alternatively or additionally, resiliency of the flat seal may provide a biasing force that biases the actuation member to the position in which the flat seal is closed.

Alternatively or additionally, the medical device may further include a spring that biases the actuation member to the position in which the flat seal is closed.

Alternatively or additionally, the proximal cap may be adapted to provide a rotational lock with a second medical device extending through the medical device hub.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 provides an illustrative example of part of a procedure for implanting an LAAC (left atrial appendage closure) device, which includes a guide catheter used in combination with an elongate dilator;

FIG. 2 is a schematic view of an illustrative guide catheter, shown in a closed configuration;

FIG. 3 is a cross-sectional view taken along the line 3-3 of FIG. 2;

FIG. 4 is an end view of the illustrative guide catheter of FIG. 2;

FIG. 5 is a schematic view of the illustrative guide catheter of FIG. 2, shown in an open configuration;

FIG. 6 is a cross-sectional view taken along the line 6-6 of FIG. 5;

FIG. 7 is an end view of the illustrative guide catheter of FIG. 5; and

FIG. 8 is a perspective view of the illustrative flat seal used in the illustrative guide catheter of FIG. 2.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A variety of medical procedures may include the use of two different medical devices that are used in combination. As an example, a first medical device may be used in combination with a second medical device in order to gain access to a particular treatment site and then to carry out an appropriate treatment at the particular treatment site. In some cases, a guidewire may be advanced through the vasculature to reach a particular treatment site. A first medical device may be advanced over the guidewire, and then a second medical device may be advanced through the first medical device. In some instances, there may be a desire to be able to advance and control the first medical device and the second medical device as an assembly, meaning that both the first medical device and the second medical device may be moved, advanced or withdrawn axially without the user being required to separately hold both the first medical device and the second medical device at the same time. In some instances, the first medical device and the second medical device may be rotated as an assembly, meaning that the first medical device and the second medical device may be rotated together without the user being required to separately hold both the first medical device and the second medical device at the same time. In some instances, there can be advantages to being able to advance or rotate the first medical device and the second medical device together, particularly when the first medical device and the second medical device have curved distal regions and/or are steerable.

An illustrative but non-limiting example of a medical procedure that may utilize a first medical device and a second medical device in combination may include reaching the left atrium in order to implant a left atrial appendage closure (LAAC) device. In particular, a first medical device such as a guide catheter may be used in combination with a second medical device such as an elongate dilator. It will be appreciated that two such medical devices may be used in combination in performing any of a variety of different medical procedures. Gaining access to the left atrium in order to deliver and implant an LAAC is merely an example of using a first medical device in combination with a second medical device.

In some instances, a guide catheter for accommodating a left atrial appendage closure (LAAC) device being advanced towards a left atrial appendage (LAA) includes an elongate shaft extending from a proximal region to a distal region and a guide catheter hub that is coupled to the proximal region of the elongate shaft. The guide catheter hub includes a hub body and a proximal cap that is secured to the hub body and that includes a proximal cap extension defining a lumen extending through the proximal cap extension. An actuation member is slidingly disposed within the hub body and includes a central bore defining a lumen extending through the actuation member and a handle extending radially outwardly from the central bore. A flat seal is disposed within the actuation member. Advancing the actuation member towards the proximal cap causes the proximal cap extension to extend through the flat seal.

In some cases, the actuation member may be movable between a first position in which the flat seal is closed and a second position in which the flat seal is open. In some cases, the actuation member may be biased to the first position. The guide catheter may further include a spring that biases the actuation member to the first position. In some cases, instead of including a separate spring, the resiliency of the flat seal may itself provide a biasing force to bias the actuation member to the first position. In some cases, the lumen extending through the proximal cap extension may be fluidly coupled to and axially aligned with the lumen extending through the actuation member when the actuation member is in the second position. The guide catheter may further include a distal lumen extending through the hub body, and the distal lumen may be fluidly coupled to and axially aligned with the lumen extending through the actuation member. In some cases, the proximal cap may be adapted to provide a rotational lock with a dilator.

In some instances, a guide catheter for accommodating a left atrial appendage closure (LAAC) device being advanced towards a left atrial appendage (LAA) may include an elongate shaft extending from a proximal region to a distal region and a guide catheter hub that is coupled to the proximal region of the elongate shaft. The guide catheter hub includes a hub body and a proximal cap that is secured to the hub body. The proximal cap includes a proximal cap extension defining a lumen extending through the proximal cap extension. An actuation member is slidingly disposed within the hub body and includes a central bore defining a lumen extending through the actuation member that is axially aligned with the lumen extending through the proximal cap extension and a handle extending radially outwardly from the central bore. A flat seal is disposed within the actuation member. The actuation member is biased to a closed position in which the flat seal prevents the lumen extending through the actuation member from being fluidly coupled with the lumen extending through the proximal cap extension.

In some cases, advancing the actuation member towards the proximal cap may cause the proximal cap extension to extend through the flat seal and fluidly couple the lumen extending through the actuation member with the lumen extending through the proximal cap extension. The guide catheter may further include a distal lumen extending through the hub body, and the distal lumen may be fluidly coupled to and axially aligned with the lumen extending through the actuation member. In some cases, the actuation member may be movable from a closed position in which the flat seal is closed to an open position in which the flat seal is open. The actuation member may be adapted to return to the closed position when the handle is released. In some cases, the resiliency of the flat seal may provide a biasing force that biases the actuation member to the closed position. In some cases, the guide catheter may further include a spring that biases the actuation member to the closed position. In some cases, the proximal cap may be adapted to provide a rotational lock with a second medical device extending through the guide catheter.

In some instances, a medical device includes an elongate shaft extending from a proximal region to a distal region and a medical device hub that is coupled to the proximal region of the elongate shaft. The medical device hub includes a hub body including a distal lumen extending through the hub body and a proximal cap that is secured to the hub body. The proximal cap includes a proximal cap extension defining a lumen extending through the proximal cap extension. An actuation member is slidingly disposed within the hub body. The actuation member includes a central bore defining a lumen extending through the actuation member and a handle extending radially outwardly from the central bore. A flat seal is disposed within the actuation member. The actuation member is biased to a position in which the flat seal is closed. Advancing the actuation member towards the proximal cap causes the proximal cap extension to open the flat seal.

In some cases, the resiliency of the flat seal may provide a biasing force that biases the actuation member to the position in which the flat seal is closed. In some cases, the medical device may further include a spring that biases the actuation member to the position in which the flat seal is closed. In some cases, the proximal cap may be adapted to provide a rotational lock with a second medical device extending through the medical device hub.

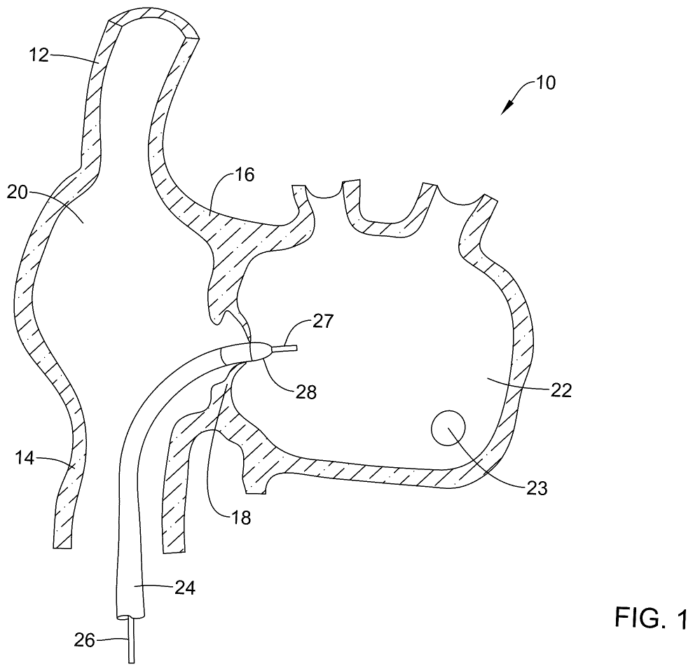

FIG. 1 provides a schematic view of a portion of a person's heart 10, including a superior vena cava 12, an inferior vena cava 14, a septum 16, an atrial septum 18, a right atrium 20 and a left atrium 22. In some cases, the left atrium 22 may include a left atrial appendage (LAA) 23. A composite medical device including a guide catheter 24 may be advanced over a guidewire 26. In some cases, the guidewire 26 may be an RF guidewire that is adapted to utilize RF (radio frequency) energy to cauterize, using a cautery tip 27. In some cases, for example, an RF guidewire may be used to form a small aperture in or near the atrial septum 18. In some cases, a guidewire may have a sharp distal end that may be used to form a puncture.

The puncture through the atrial septum 18 can be done from a position within the right atrium 20. By forming an aperture through the atrial septum 18, it is possible to reach the left atrium 22 from the relative safety of the right side of the heart. In some cases, an elongate medical device including an elongate dilator 28 may be advanced over the guidewire 26, and within the guide catheter 24. Once an aperture has been formed within the atrial septum 18, the elongate dilator 28 may be advanced over the guidewire and through the aperture in order to widen the aperture. From there, the elongate dilator 28 and guidewire 26 may be removed in order to allow a delivery device carrying an LAAC (left atrial appendage closure) device to be advanced through the guide catheter 24. A variety of devices may be advanced through the guide catheter 24 in order to reach the LAA 23. An illustrative example of a suitable LAAC device includes the Watchman FLX™ LAAC device commercially available from Boston Scientific Corporation.

In some cases, an assembly including the guide catheter 24 and the elongate dilator 28 may be advanced through the inferior vena cava 14 in order to reach the right atrium 20. It will be appreciated that this can represent a tortuous path through the vasculature. In some cases, the guide catheter 24 and/or the elongate dilator 28 may be adapted to have a curved distal end in order to facilitate steering. It will be appreciated that being able to hold the guide catheter 24 and the elongate dilator 28 from relative rotation therebetween may be beneficial in steering the devices through the anatomy. Additional details pertaining to providing a rotational lock between a first medical device and a second medical device may be found, for example, in U.S. patent application Ser. No. 18/439,047, filed Feb. 12, 2024 and entitled Multi-Part Medical Devices With Locking Mechanism, which application is incorporated by reference herein in its entirety.

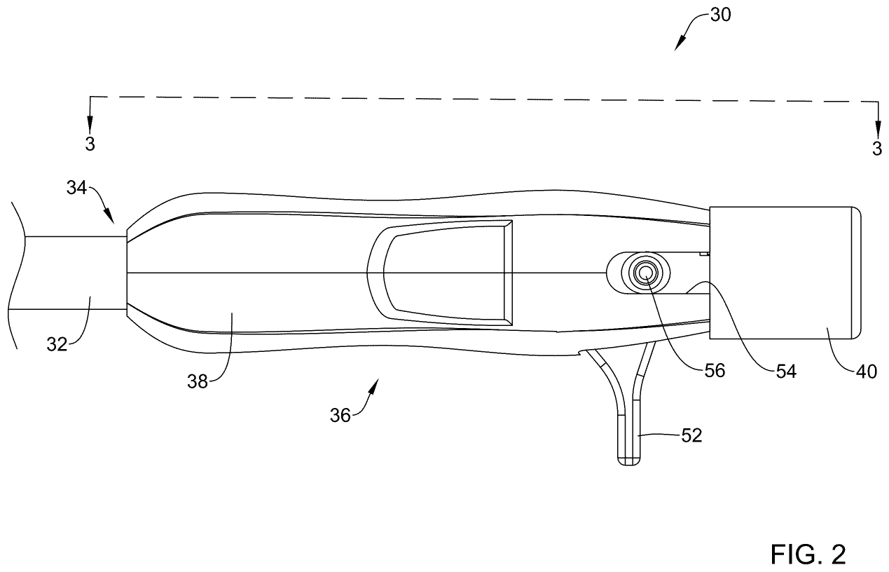

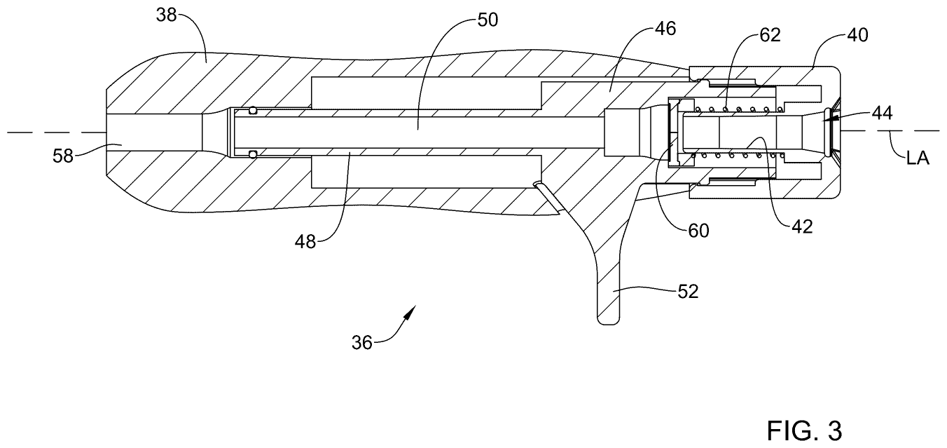

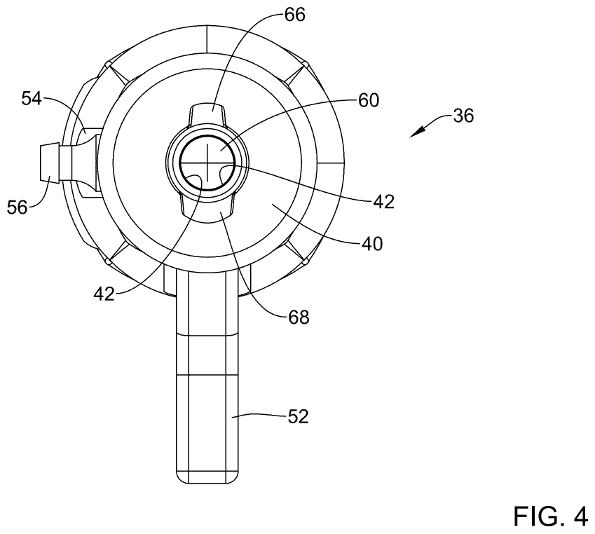

FIG. 1 shows a distal portion of the guide catheter 24. FIGS. 2 through 7 provide views of a guide catheter 30 that may be used as the guide catheter 24 in accessing the LAA 23. In some cases, the guide catheter 30 provides benefits such as improved sealing and reduced possibility of air infiltration into the guide catheter 30. FIG. 2 is a perspective view of the illustrative guide catheter 30. The guide catheter 30 may be considered as being an example of the guide catheter 24 partially shown in FIG. 1. FIG. 3 is a cross-sectional view taken along the line 3-3 of FIG. 2 and FIG. 4 is an end view. The guide catheter 30 includes an elongate shaft 32 that extends from a proximal region 34 to a distal region (as shown in FIG. 1). A guide catheter hub 36 is coupled to the proximal region 34 of the elongate shaft 32. FIGS. 2, 3 and 4 show the guide catheter hub 36 in what may be referred to as a closed position, meaning that there is no clear fluid path through the guide catheter hub 36. No clear fluid path through the guide catheter hub 36 means no air infiltration, and means that the guide catheter hub 36 is not able to accommodate any other medical devices being advanced through the guide catheter 30.

The guide catheter hub 36 includes a hub body 38 and a proximal cap 40 that is secured to the hub body 38. The proximal cap 40 includes a proximal cap extension 42 that defines a lumen 44 extending through the proximal cap extension 42. An actuation member 46 is slidingly disposed within the hub body 38. The actuation member 46 includes a central bore 48 that defines a lumen 50 extending through the actuation member 46. It can be seen that the lumen 50 extending through the actuation member 46 is axially aligned with the lumen 44 extending through the proximal cap extension 42. The actuation member 46 includes a handle 52 that extends radially outwardly from the central bore 48. In some cases, a user grasping the guide catheter hub 36 in their hand may be able to use their thumb, for example, to push the handle 52 proximally. As will be discussed, pushing the handle 52 proximally moves the guide catheter 30 towards what may be referred to as an open position, meaning that there is a clear fluid path through the guide catheter. A clear fluid path through the guide catheter hub 36 means that the guide catheter hub 36 is able to accommodate any other medical devices being advanced through the guide catheter 30.

In some cases, the hub body 38 includes an elongate slot 54 that is adapted to accommodate movement of a Luer fitting 56 that is coupled with the actuation member 46. The Luer fitting 56 may be used to connect a source of fluid (such as saline) to the guide catheter hub 36. In some cases, the Luer fitting 56 may be fluidly coupled with the lumen 50 extending through the central bore 48 of the actuation member 46.

In some cases, the hub body 38 includes a distal lumen 58 extending through part of the hub body 38. As can be seen, the distal lumen 58 is axially aligned with a longitudinal axis LA of the guide catheter hub 36. In some cases, as shown, the lumen 50 extending through the central bore 48 of the actuation member 46 is axially aligned with the longitudinal axis LA of the guide catheter hub 38. In some cases, as shown, the lumen 44 extending through the proximal cap extension 42 is axially aligned with the longitudinal axis LA of the guide catheter hub 38. The distal lumen 58 is fluidly coupled with the lumen 50 extending through the central bore 48 of the actuation member 46. The central bore 48 of the actuation member 46, as shown, is not fluidly coupled with the lumen 44 extending through the proximal cap extension 42. As shown, a flat seal 60 that is disposed within the actuation member 46 prevents the bore 48 of the actuation member 46 from being fluidly coupled with the lumen 44 extending through the proximal cap extension 42. This can also be seen in FIG. 4, where a portion of the flat seal 60 is visible through the lumen 44 extending through the proximal cap extension 42.

In some cases, the guide catheter 30 includes a spring 62 that is disposed between the proximal cap 40 and the actuation member 46 in order to bias the actuation member 46 in the closed position as shown in FIGS. 2, 3 and 4. In some instances, the spring 62 may be excluded, particularly if the flat seal 60 is made of an elastomeric material that itself can provide a biasing force sufficient to bias the actuation member 46 into the closed position.

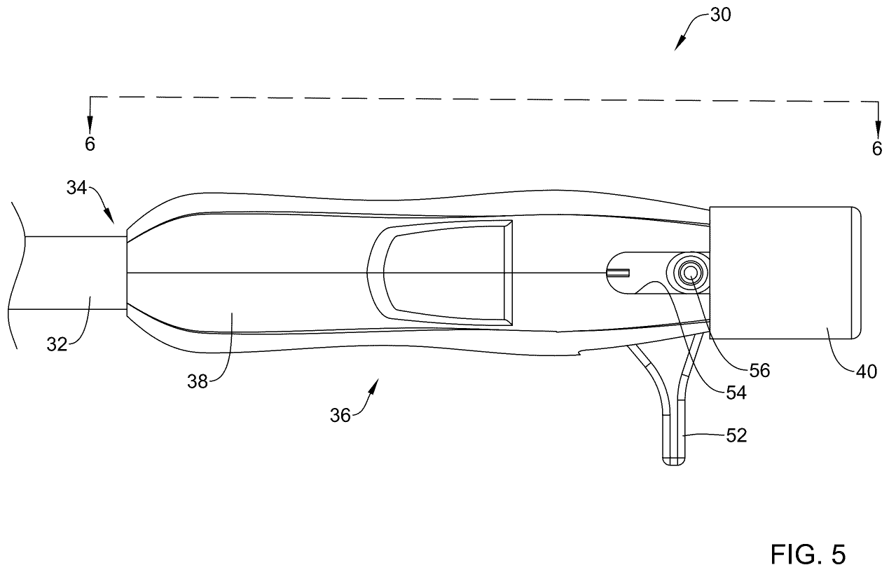

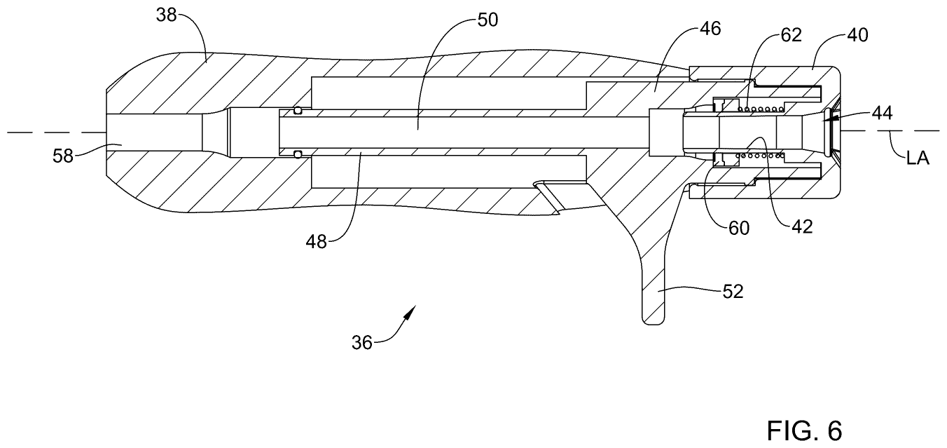

As noted, FIGS. 2, 3 and 4 show the actuation member 46 in the closed position in which the proximal cap extension 42 does not engage or penetrate the flat seal 60. Urging the handle 52 in a proximal direction (towards the proximal cap 40) causes the actuation member 46 to move from the closed position towards and into the open position. As can be seen in FIG. 5, the handle 52 has been moved towards the proximal cap 40. The Luer fitting 56 has translated within the elongate slot 54. In viewing the cross-sectional view shown in FIG. 6, it can be seen that the spring 62 has been compressed, and the flat seal 60 has been pushed onto the proximal cap extension 42 of the proximal cap 40 such that the proximal cap extension 42 extends through the flat seal 60.

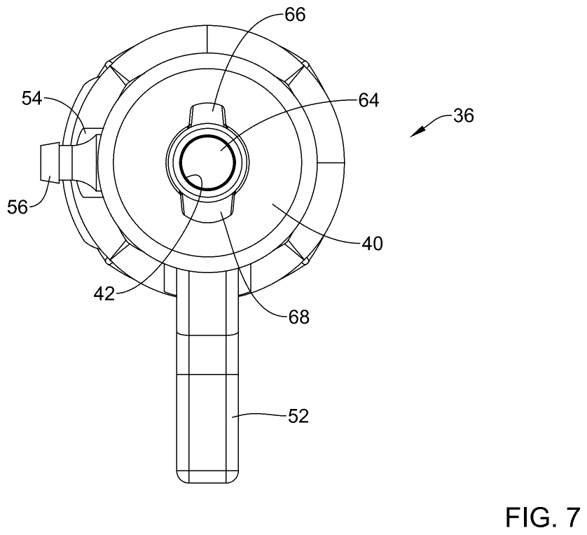

Now, the lumen 50 extending through the central bore 48 is fluidly coupled with the lumen 44 extending through the proximal cap extension 42. Because the distal lumen 58 within the hub body 38 is also fluidly coupled with the lumen 50 extending through the central bore 48, and all three lumens 44, 50 and 58 are axially aligned, this means that there is an open path through the guide catheter hub 36 that allows other medical devices (such as but not limited to a dilator such as the elongate dilator 28 shown in FIG. 1) to be advanced through the guide catheter hub 36 and into the elongate shaft 32. In looking at FIG. 7, it can be seen that there is now a void 64 that extends all the way through the guide catheter hub 36. The void 64 corresponds to an interior of the lumen 44 extending through the proximal cap extension 42, an interior of the lumen 50 extending through the central bore 48 and an interior of the distal lumen 58.

In some cases, the proximal cap 40 may be adapted to help provide a rotational lock with other medical devices to be advanced within the guide catheter 30. As an example, the proximal cap 40 may be adapted to help provide a rotational lock with a dilator such as the elongate dilator 28 (FIG. 1). As can be seen in FIGS. 4 and 7, the proximal cap 40 includes a first detent 66 and a second detent 68 disposed on either side of the proximal cap extension 42. In some cases, the first detent 66 and the second detent 68 may be the same size and shape. In some cases, as shown, the first detent 66 and the second detent 68 may have different sizes and/or different shapes in order to provide a single correct insertion position for the elongate dilator 28. While not shown, the elongate dilator 28 may include corresponding protrusions that fit into the first detent 66 and the second detent 68. Further details regarding a possible rotational lock between the guide catheter hub 36 and the elongate dilator 28 may be found in the previously cited and incorporated U.S. patent application Ser. No. 18/439,047.

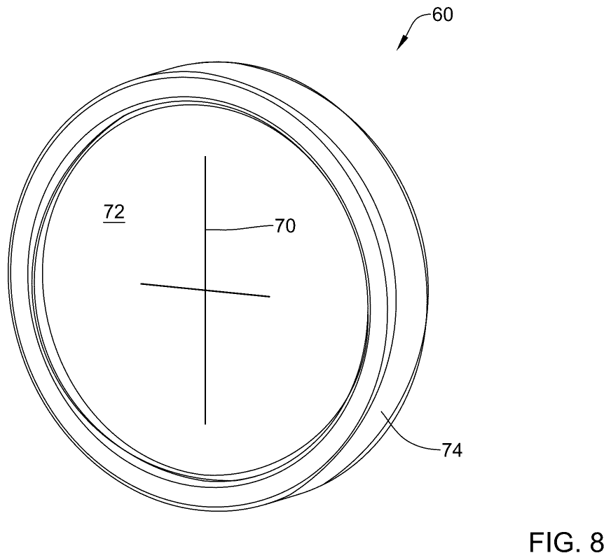

FIG. 8 is a perspective view of the flat seal 60. As can be seen, the flat seal 60 includes a slit 70 that extends across a face 72 of the flat seal 60. The flat seal 60 may include a single slit 70, or may include two or more slits 70 that intersect each other. The flat seal 60 has a periphery 74 that extends around the flat seal 60 and that provides a circular profile that allows the flat seal 60 to fit within the actuation member 46. The slit 70 is normally closed but can be opened by extending something through the slit 70. The flat seal 60 may be opened by advancing the actuation member 46, and thus the flat seal 60, proximally towards and into engagement with the flat seal 60. The flat seal 60 may be formed of any suitable elastomeric material. In some cases, the flat seal 60 may be formed of an elastomeric polymer such as silicone.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel alloys; combinations thereof; and the like; or any other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A guide catheter for accommodating a left atrial appendage closure (LAAC) device being advanced towards a left atrial appendage (LAA), the guide catheter comprising:
an elongate shaft extending from a proximal region to a distal region;
a guide catheter hub coupled to the proximal region of the elongate shaft, the guide catheter hub including:
a hub body;
a proximal cap secured to the hub body, the proximal cap including a proximal cap extension defining a lumen extending through the proximal cap extension;
an actuation member slidingly disposed within the hub body, the actuation member including:
a central bore defining a lumen extending through the actuation member; and
a handle extending radially outwardly from the central bore;
a flat seal disposed within the actuation member;
wherein advancing the actuation member towards the proximal cap causes the proximal cap extension to extend through the flat seal.

2. The guide catheter of claim 1, wherein the actuation member is movable between a first position in which the flat seal is closed and a second position in which the flat seal is open.

3. The guide catheter of claim 2, wherein the actuation member is biased to the first position.

4. The guide catheter of claim 3, further comprising a spring that biases the actuation member to the first position.

5. The guide catheter of claim 3, wherein resiliency of the flat seal provides a biasing force to bias the actuation member to the first position.

6. The guide catheter of claim 2, wherein the lumen extending through the proximal cap extension is fluidly coupled to and axially aligned with the lumen extending through the actuation member when the actuation member is in the second position.

7. The guide catheter of claim 6, further comprising a distal lumen extending through the hub body, and the distal lumen is fluidly coupled to and axially aligned with the lumen extending through the actuation member.

8. The guide catheter of claim 1, wherein the proximal cap is adapted to provide a rotational lock with a dilator.

9. A guide catheter for accommodating a left atrial appendage closure (LAAC) device being advanced towards a left atrial appendage (LAA), the guide catheter comprising:
an elongate shaft extending from a proximal region to a distal region;
a guide catheter hub coupled to the proximal region of the elongate shaft, the guide catheter hub including:
a hub body;
a proximal cap secured to the hub body, the proximal cap including a proximal cap extension defining a lumen extending through the proximal cap extension;
an actuation member slidingly disposed within the hub body, the actuation member including:
a central bore defining a lumen extending through the actuation member that is axially aligned with the lumen extending through the proximal cap extension;
a handle extending radially outwardly from the central bore; and
a flat seal disposed within the actuation member;
wherein the actuation member is biased to a closed position in which the flat seal prevents the lumen extending through the actuation member from being fluidly coupled with the lumen extending through the proximal cap extension.

10. The guide catheter of claim 9, wherein advancing the actuation member towards the proximal cap causes the proximal cap extension to extend through the flat seal and fluidly couple the lumen extending through the actuation member with the lumen extending through the proximal cap extension.

11. The guide catheter of claim 10, further comprising a distal lumen extending through the hub body, and the distal lumen is fluidly coupled to and axially aligned with the lumen extending through the actuation member.

12. The guide catheter of claim 9, wherein the actuation member is movable from a closed position in which the flat seal is closed to an open position in which the flat seal is open.

13. The guide catheter of claim 12, wherein the actuation member is adapted to return to the closed position when the handle is released.

14. The guide catheter of claim 12, wherein resiliency of the flat seal provides a biasing force that biases the actuation member to the closed position.

15. The guide catheter of claim 9, further comprising a spring that biases the actuation member to the closed position.

16. The guide catheter of claim 9, wherein the proximal cap is adapted to provide a rotational lock with a second medical device extending through the guide catheter.

17. A medical device, comprising:
an elongate shaft extending from a proximal region to a distal region;
a medical device hub coupled to the proximal region of the elongate shaft, the medical device hub including:
a hub body including a distal lumen extending through the hub body;
a proximal cap secured to the hub body, the proximal cap including a proximal cap extension defining a lumen extending through the proximal cap extension;
an actuation member slidingly disposed within the hub body, the actuation member including:
a central bore defining a lumen extending through the actuation member;
a handle extending radially outwardly from the central bore; and
a flat seal disposed within the actuation member;
wherein the actuation member is biased to a position in which the flat seal is closed; and
wherein advancing the actuation member towards the proximal cap causes the proximal cap extension to open the flat seal.

18. The medical device of claim 17, wherein resiliency of the flat seal provides a biasing force that biases the actuation member to the position in which the flat seal is closed.

19. The medical device of claim 17, further comprising a spring that biases the actuation member to the position in which the flat seal is closed.

20. The medical device of claim 17, wherein the proximal cap is adapted to provide a rotational lock with a second medical device extending through the medical device hub.
